(19) United States
(12) Patent Application Publication
(10) Pub. No.: US 2013/0211143 A1
(43) Pub. Date: Aug. 15, 2013

(54) Method and an Apparatus for Producing Organic Solvents and Alcohols by Microbes

(71) Applicant: Tom Granstrom, Rajamaki (FI)

(72) Inventor: Tom Granstrom, Rajamaki (FI)

(73) Assignee: AALTO UNIVERSITY FOUNDATION, AALTO (FI)

(21) Appl. No.: 13/809,003

(22) PCT Filed: Jul. 8, 2011
(86) PCT No.: PCT/FI2011/050644

§ 371 Date: Mar. 12, 2013

(30) Foreign Application Priority Data
Jul. 8, 2010 (FI) 20105782

(51) Int. Cl. C12P 7/28; C12P 7/10; C07C 31/08; C12P 7/16; C07C 49/08; C07C 31/12

(52) U.S. Cl. CPC C12P 7/28; C07C 49/08; C07C 31/12; C07C 31/08; C12P 7/16; C12P 7/10
USPC 568/382; 568/840; 435/179; 435/289.1; 435/150; 435/160; 435/165

(57) ABSTRACT

The present invention relates to a cell retaining matrix (CRB) for producing solvents and alcohols and an apparatus comprising said bio-column. The invention also relates to the method for producing solvents and alcohols by using said cell retaining matrix (CRB). Said cell retaining biomatrix (CRB cellulosic fibers, selected from the group consisting wood cellulosic fibers, pulp cellulosic fibers, vegetable cellulosic fibers, such as mechanical pulp, dissolving pulp, lignocellulosic fibers, and cellulosic fibers originated from vegetable peels, and at microbes, which have been immobilized into said cellulosic fibers, and which can save their biological activity in cell retaining biomatrix (CRB).

The present invention relates to a bio-column for producing solvents and alcohols and an apparatus comprising said bio-column. The invention also relates to the method for producing solvents and alcohols by using said bio-column.

Production of organic solvents and alcohols is perhaps the most important biological processes area. For example acetone-butanol-ethanol (ABE) fermentation by Clostridium acetobutylinum has nowadays received considerable attention. The Weizmann starch process is one of the first well known processes using Clostridium acetobutylinum to microbiologically prepare acetone and butanol using an anaerobic fermentation process. C. acetobutylinum and other Clostridium species can digest for example sugar, starch, lignin and other biomass directly into propionic acid, butanol, ether and glycerin. Historically industrial scale fermentations were performed for acid and solvent production prior to the rise of the petrochemical industry. Since the 1960's the ABE industry has declined. Recent progresses in the fields of bioprocessing and biotechnology have resulted in a renewed interest in the fermentation production of chemicals and fuels, especially butanol. With continuous fermentation technology, butanol can be produced at higher yields, concentrations and production rates.

Acetone/isopropanol, butanol, ethanol are applicable as liquid transport biofuels in combustion engines. Butanol is a preferred transportation fuel because its energy content is close to that of gasoline (26.9 vs. 32 MJ/L), it can be stored under humid conditions because of its low miscibility with water, and is compatible with the existing gasoline supply infrastructure. Despite of the extensive knowledge there are still unsolved problems preventing the large scale utilization of Clostridia spp. in the industry. These include low yields, consecutive acid production and solvent accumulation stages and narrow substrate preference of Clostridia spp.

ABE fermentations processes are mainly optimized using starch or molasses as a feedstock (Mitchell, 1998). Clostridia strain harbors all the required amylolytic enzyme activities for complete starch degradation and subsequent fermentation to end products (Nimcevic et al. 1998). However, any feed stock applicable for human nourishment cannot be listed as a 2^{nd} generation biofuel nor fulfill the requirements of ethical production of bioenergy from renewable biomass. Forest or plant residue or industrial waste streams would constitute more environmentally acceptable and available feed stocks. The liquor hydrolysates prepared from softwood, hardwood and de-inked paper consisted mostly of xylose, mannose and galactose sugars depending on the wood species and the selected hydrolysis method (Rakkolainen et al. 2010). In the past butanol was produced from the hydrolyzed agricultural lignocellulosic waste in the Russian ABE plants. The pentose hydrolysate was generated using a 1% H₂SO₄ solution at 115-125° C. for 1.5-3 hours with the average solvent yield of 20-32% (Zverlov et al. 2006). The fermentation time is approximately 20-25 hours with the preferred substrate i.e. starch, but using lignocellulose hydrolysates as feed stock it will be an essentially longer (Ezeji and Blaschek, 2008).

The most difficult challenge in an industrial ABE fermentation is the low yield of solvents. Even with the most preferred substrate, i.e. starch and molasses, the total yield of solvents is around 30-32% and the highest concentration of butanol reaches 10-15 g/L in traditional batch fermentation (Walton and Martin, 1979). There have been reports where C. acetobutylicum solvent tolerance has been increased by serial enrichment methods (Lin and Blaschek (1983). However, the reproducibility of these kinds of results with different growth substrate and conditions is weak. In some cases the serial transfer will end up in Clostridia spp. completely losing their ability to produce solvents. This degeneration has been attributed to the loss of megaplasmid containing the solventogenetic genes (Cornillot et al. 1997).

C. acetobutylicum is a sporulating, Gram-positive microbe and the binding region of the Spo0A gene is located in the promoter region of the solventogenetic genes (Thormann et al., 2002) acting as a transcriptional regulator of sporulation and solvent production (Harris et al., 2002). Due to the complex life cycle and metabolism of C. acetobutylicum new bacterial hosts for butanol production has been developed recently. The recombinant Lactobacillus brevis strain containing the clostridial genes of butanol pathway (crt, bcd, etfB, etfA, and hbd) was able to synthesize up to 300 mg l⁻¹ or 4.1 mM of butanol on a glucose-containing medium (Berezina et al., 2010).

Two stage chemostat set up has been studied as one of the options for higher productivities in ABE fermentation (Mutschlechner et al., 2000; Bahl et al., 1982). Chemostat set up will allow constant removal of butanol, which is the prerequisite for high solvent productivity and product yield. It is likely that free cell suspension cultivations do not achieve industrially applicable solvent productivity in particular concerning the production of liquid transport biofuels from lignocellulosic biomass. ABE fermentation offers one possibility for future biorefineries to implement the necessary bioenergy resolutions from lignocellulosic biomass. This requires new improvements in the bioprocess engineering and also in strain development research. The metabolically engineered strains have to be viable and tolerant for different inhibitors present in the real hydrolysates. The bioprocess engineering has to take into consideration very large volumes required to fulfill the consumption of liquid transport fuels.

Number of attempts has been reported to use so called biomass retaining or recycling methods. Tashiro et al. (2005) achieved the ABE productivity of 7.55 g⁻¹ L⁻¹ h⁻¹ at an ABE concentration of 8.58 g/l at dilution rates of 0.11 h⁻¹ using continuous culture with cell-recycling. Qureshi and Blaschek (2004) used brick pieces to immobilize cells into a column and achieved high ABE productivities compared to the free cell suspensions, but the column suffered from technical problems. Ennis and Maddox (1989) studied the solvent production with a continuous bioreactor where cells were recycled using a cross-flow microfiltration (CFM) membrane unit. The solvent productivity of 2.92 kg/m³ with a yield of 0.31 kg kg⁻¹ was reported.

Berezina et al. (2008) reported low cellulase activities of different strains of C. acetobutylicum against carboxymethylcellulose, and crystalline and amorphous celluloses. According to López-Contreras et al. (2004) C. acetobutylicum does not degrade cellulose, but low level of induction of cellulase activity occurs during growth on xylose or lichenan.

ABE fermentation using Clostridium species is a process that requires two steps. In the first stage, acidogenesis, acetic, propionic, lactic and butyric acids are produced. In the second stage, solventogenesis, acetone, butanol, ethanol and isopropanol are produced. Cells from the acidogenetic or solventogenetic stage can be loaded into a column, substrate flow is then pumped through the column and the solvents are produced. The process must be effected under fully anaerobic conditions.

WO 2009/126795 A discloses a process, where there are at least two bioreactors arranged in a series or in parallel for the continuous production of butanol using Clostridium species which are immobilized on a solid support. Additional feeding of enzymes is needed to break down natural polymers into simpler constituents that can be assimilated by the microorganisms.

WO 81/01012 A discloses a process for the microbiological preparation of solvents. Immobilized, non-growing cells of solvent producing strains of Clostridium species are used in a two-step ABE-process, where cell mass is first grown in optimal conditions and then the cells are immobilized by various methods.

EP 0282474 (A1) discloses a continuous ABE-I production process, where the first step consist of continuous cultivation of the bacteria and the second step, where the bacteria are immobilized on a carrier material, consists of the product formation. The second step is carried out continuously or in batches.

### GENERAL DESCRIPTION

The object of the present invention is to provide a biomatrix, which provides a technically simple process for producing solvents and alcohols from different substrates.

Another object of the present invention is to provide an apparatus and method for producing organic solvents and alcohols from different substrates by microbes.

To achieve this object the invention is characterized by the features that are enlisted in the independent claims. Other claims some represent preferred embodiments of the invention.

The invention is based on a cell retaining biomatrix, which comprises cellulosic fibers, and microbes, which have been immobilized into said cellulosic fibers, and which can save their biological activity in cell retaining biomatrix (CRB). Preferable biologically active microbes can save their biological activity at least for 14 days in cell retaining biomatrix (CRB).

It has now been surprisingly found that using a cell retaining cellulosic fibers as a biomatrix with biologically active microbes, leads to a better yield and increases the production rate of organic solvents and alcohols. At same time substrate(s) and optionally also degradable cell retaining biomatrix are converted to organic acids and alcohols by selected microbes. This simplifies the process and improves economy of production of organic acids and alcohols.

The cell retaining biomatrix is preferably selected from the group consisting wood cellulosic fibers, pulp cellulosic fibers, vegetable cellulosic fibers, such as mechanical pulp, dissolving pulp, lignocellulosic fibers and cellulosic fibers originated from vegetable peels. Cellulosic fibers are used as a column filling prepared e.g. from wood biomass by ethanol-water-SO₂ cooking. C. acetobutylicum is able hydrolyse cellulose since it contains the operon for the cellulosome genes, but the cellulose activity is inducible depending on the conditions. It is preferably to use highly degraded cellulosic pulp as a cell retaining biomatrix in the bio-column. Cell retaining biomatrix is optionally degraded and used as a nutrient source by the microbes over time and its distribution is uniform. The use of this type of bio-column is also highly ecological, since pulp is fully biological and degradable, and the remaining matrix can be washed “clean” from the cells, after which it is possible to recycle or re-use it depending on the level of exposure of cell metabolism during the production phase. The use of wood biomass, preferably pulp, is also highly cost-efficient, because it is generally readily available and typically low in price.

According to one embodiment the cell retaining biomatrix is in the form of a sheet or a mat or a net. The structure of cell retaining biomatrix can vary. ABE fermentation by Clostridium species requires two steps, hence the different layers/zones in the biomatrix. There are also different ways to treat wood fibers when producing the biomatrix; it can for example be made into a mat or a sheet, depending on the microbe used or the scale of the process.

According to one embodiment the cell retaining biomatrix is in the form of individual fibers or flocs.

According to one embodiment, the cell retaining biomatrix further comprises a support structure and/or an effluent splitter. This essentially improves fluid in biomatrix and leads to better yield of solvents and alcohols from different substrates.

According to one embodiment, the cell retaining biomatrix further comprises polypropylene or polyethylene.

Regardless of the form of the cell retaining biomatrix (a sheet, a mat or individual fibers), the cell retaining biomatrix can be preferably rolled with a supporting net that can be made of polypropylene, polyethylene or some other inert material for microbiological reactions. Fibers or other biomatrix are placed with the supporting matrix on top of each other as layers and rolled into discs with desired length and diameter. The thickness of each layer and the ratio of each material can vary depending on the used microbes.

Biological Activity can be monitored my measuring composition of different substrates and/or cellulosic fibers, and/or by measuring viability of selected microbes. It can be e.g. monitored by measuring degradation of cellulosic material and amounts of nutrient and concentrations in fluids and masses.

The microbes can be immobilized into the cell retaining biomatrix by the general methods known in the art. These include for example pumping cell suspension through the cell retaining matrix until column is saturated with cells. Matrix can be mixed with high cell concentration suspension and packed into the column.

According to one embodiment the said microbe is selected from the group consisting Clostridia species, such as, C. acetobutylicum, C. butyricum, C. beijerinckii, C. saccharobutylacetonicum and C. saccharobutylicum, or Lactobacillus species (lactic acid bacteria) such as L. plantarum, L. brevis, L. fermentum, L. sanfranciscensis, L. buchneri, L. collinoides, L. rhamnosus and L. bulgaricus. Said microbe can be e.g. in wild type, mutant type and genetically modified type microbe.

Another aspect of the invention comprises an apparatus for producing organic solvents and alcohols from different substrates by microbes. Apparatus according to the invention comprises at least a bio-column comprising wood fibers as cell retaining biomatrix, which is degradable and usable as a nutrient source for selected microbes.

According to one embodiment, the apparatus further comprises a separate or integrated cell growing unit having a feeding device for feeding first substrate and an adjusting device for controlling growth conditions of cells of selected microbe(s) in the first solution in said integrated cell growing unit.

According to another embodiment, the apparatus further comprises a separate or an integrated fermentation adjusting unit having an adjusting device for adjusting condition in second solution to favor production of organic solvents and alcohols by cells of selected microbes. Preferably said adjusting unit comprises a feeding device for feeding cells and/or first solution from said integrated cell growing unit and an adjusting device for adjusting condition in second solution to favor production of organic solvents and alcohols by cells of selected microbes. Preferably said adjusting unit comprises a additional feeding unit for feeding additional substrate to said fermentation adjusting unit.

According to another embodiment, the apparatus further comprises a separate or an integrated solution recovering unit for recovering third solution from the bio-column comprising organic solvents and alcohols.

According to one embodiment, the apparatus further comprises a cell return unit for recovering cells, solution and/or fibers originated from the bio-column, from the substrate and/or from the solution recovering unit. Optionally at least part of those cells are returned to the integrated cell growing unit, to the fermentation adjusting unit and/or to the bio-column. This has the advantage reducing the costs of growing the cells. Also cells can be returned to the first unit for regeneration of the cell activity in the most optimum conditions.

The invention also comprises a method for producing organic solvents and alcohols from different substrates comprising at least the following steps:
feeding substrate (SU1, SU2) to the cell retaining biomatrix (CRB) according to of the invention, and
producing organic solvents and alcohols by microbes by feeding substrate to the cell retaining biomatrix (CRB), and
recovering solution comprising organic solvents and alcohols.

The invention is also preferably based on the method for producing organic solvents and alcohols from different substrates by microbe(s), which comprises at least the following steps:

a) growing cells of microbes on a continuous, batch or fed batch mode in first solution by feeding first substrate and adjusting condition of said first solution for optimal growth conditions of cells of microbes;
b) optionally adjusting condition of grown cells in second solution on a continuous, batch or fed-batch mode to favor production of organic solvents and alcohols by cells of microbes; and
c) introducing growing or adjusted cells from step a) and/or b) into the bio-column comprising the cell retaining biomatrix,

Preferable at least partly degradable and usable as a nutrient source for adjusted microbes originated from the fermentation adjusting unit.

The cell retaining biomatrix is preferably selected from the group consisting wood cellulosic fibers, pulp cellulosic fibers, vegetable cellulosic fibers, such as mechanical pulp, dissolving pulp, lignocellulosic fibers and cellulosic fibers originated from vegetable peels.

According to one embodiment, the method further comprises the steps of

d) producing organic solvents and alcohols by adjusted cells of microbes by feeding second substrate to the cell retaining biomatrix; and
e) recovering third solution from step d) comprising organic solvents and alcohols.

According to one embodiment of the method the cells of microbes are recovered from step d) and/or from step e). Optionally at least part of those cells are fed back to step b).

According to one embodiment of the method the feeding of the second substrate in d) is initiated once the cell retaining biomatrix is saturated with cells of microbes. This further improves yield and increases production rate because the biomatrix is in full use.

According to one embodiment of the method the amount of cells is controlled by measuring optical density value of solutions. This is an advantageous and reliable method for measuring amount of cells. It also gives the means to control the process more accurately and more precisely.

According to one embodiment of the method substrate is selected from the group consisting of monomeric and oligomeric sugars, substrate originated from wood biomass and lignocellulosic biomass and substrate originated from vegetable peels, such as sulphite spent liquor (SSL), POME, and/or EFB.

By adjusting the degree of polymerization of pulp by controlling for example sulphur dioxide concentration in the cooking of lignocellulosic biomass it can be made more degradable for cell metabolism during the exposure of cell contact and vice versa. The composition of the pulp can be varied from individual fibers to uniform sheets according to the column structure.

According to one embodiment of the method the growth conditions in step a) and step b) are optimized by controlling pH value, the growth rate, feeding rate, temperature, sugar composition and substrate concentration.

According to one embodiment of the method the pH value of the first solution in step a) is in the range of 3.5-4.5 and pH value of the second solution in step b) is in the range of 4.5-6.5. In the batch mode the pH is in the range of 3.5-6.5.

According to one embodiment of the method in step e) the third solution, comprising organic solvents and alcohols, is simultaneously recovered when feeding second substrate to cell retaining biomatrix in step d). This simultaneous recovery of the solution either reduces or totally eliminates the possible end product inhibition of the microbial cells.

The invention is also based on the use of the bio-column for producing organic solvents and alcohols as feedstock for liquid fuels, chemicals, polymers and biomaterials by the method.

According to one embodiment of the present invention organic solvents and alcohols, such as acetone, butanol, ethanol and isopropanol, are produced by a biomatrix according to the invention. According to one embodiment of the use, the said microbe is Clostridia species or Lactobacillus species. Preferably feeding substrate is a substrate selected from the group consisting of monomeric and oligomeric sugars, substrate originated from wood biomass and lignocellulosic biomass and substrate originated from vegetable peels, such as sulphite spent liquor (SSL), POME, and EFB. According to one embodiment of the use, the organic solvents and alcohols produced are selected from the group consisting acetone, butanol, ethanol and isopropanol. Other products include acetic acid, butyric acid, lactic acid, acetaldehyde butyraldehyde, succinic acid and propionic acid.

### DETAILED DESCRIPTION OF THE INVENTION

Some embodiments of the invention will be explained next in more detail in examples.

### Organism, Maintenance and Inoculum Preparation

Clostridia acetobutylicum B 5313 (DSM 792, ATCC 824) was obtained from Russian National Collection of Industrial Microorganisms at the Institute of Genetics and Selection of Industrial Microorganisms (Moscow, Russia). Frozen stock cultures containing 20% (w/v) glycerol were stored in 2 ml ampoules at −70° C. Inoculum for fermentation was prepared in 125-ml air-tight, anaerobic glass flasks and grown overnight on MSS-medium (Berezina et al., 2008) at 37° C. MSS-medium contained 5 g/l of yeast extract powder (Scharlau), 60 g/l of glucose (VWR), 0.8 g/l K₂HPO₄₂HPO₄ (J.T. Baker), 0.01 g/l p-aminobenzoic acid (Fluka), 0.5 g/l cysteine-hydrochloride (Aldrich), 3 g/l CH₃COONH₄ (Merck), 1 g/l KH₂PO₄ (J.T. Baker), 1 g/l MgSO₄.7H₂O (J.T. Baker), and 0.05 g/l FeSO₄.7H₂O (Merck).

### Chemostat Cultivations

Two-stage chemostat cultivations were carried out in two 1-liter fermenters (Braun Biostat Q) F1, F2 on MSS medium (20 g/l of glucose concentration) or SOL medium at 37° C. with a stirrer speed 50 rpm. SOL medium was prepared according to Liubimova et al. (1993) containing: 1 g/l tryptone (Lab M), 1 g/l yeast extract powder (Scharlau), 60 g/l glucose (VWR), 0.7 g/l K₂HPO₄ (J.T. Baker), 0.5 g/l cysteine-hydrochloride (Aldrich), 0.01 g/l NaCl (J.T. Baker), 3 g/l CH₃COONH₄ (Merck), 0.7 g/l KH₂PO₄ (J.T. Baker), 0.1 g/l MgSO₄.7H₂O (J.T. Baker), 0.02 g/l MnSO₄.H₂O (Merck), and 0.015 g/l FeSO₄.7H₂O (Merck). Alternatively the sugar mixture of glucose 6.3 g/l; arabinose 2.23 g/l; galactose 6.43 g/l; mannose 22.85 g/l; xylose 7.51 g/l were used. The medium was autoclaved for 20 min at 121° C. The culture pH was set at 4.6 and 5.1 in the F1 and F2 unit respectively and it was controlled with 4 M NaOH. The dilution rate was adjusted to 0.05 h⁻¹ and 0.1 h⁻¹ consecutively. The working volume of 500 ml was kept constant by removing the effluent with peristaltic pumps from both fermentation units (Peristaltic Pump P-3, Pharmacia Fine Chemicals). Samples for biomass and HPLC were taken on a two consecutive days to confirm the steady state conditions.

### Cell Dry Weight Measurements

Culture samples (40 ml) were centrifuged (Eppendorf, Centrifuge 5804R) at 5000 rpm for 10 minutes, washed with Milli-Q water, and then dried in an oven at 80° C. for 24-hours (Heraeus) in glass Petri plates, which were weighed before adding the sample. Once dry, the plates were weighed.

### Cell Retaining Column

Pharmacia column (XK26) was filled with water saturated cellulosic fibers supported by a plastic net. 50 g w/w spruce chips fibers were rolled together into a tubular form with a plastic net and inserted into column. The bed height was 20.7 cm and the corresponding void volume was 102 cm³. The column was sterilized overnight with ethanol and the column volume of 4.6 cm³ was determined by flushing the column with a growth medium. Actively growing and producing Clostridia cell mass was loaded into the bio-column by pumping cell suspension with a high flow rate through the matrix. The out flowing cell mass was returned to the F2 bioreactor unit. Cell mass retention was monitored by the decreasing optical density value at 600 nm. After the bio-matrix was saturated with cells the loading was stopped. The substrate solution feeding was initiated from the separate substrate bottle placed in the water bath at 37° C. from the bottom direction. The ABE-solution product was collected from the top of the column. When productivity was decreased the cell loading was repeated.

### SEW Fractionation of Spruce Chips to Produce Pulp

Industrial spruce chips screened to 2-4 mm thickness and then air-dried were used as a raw material for pulping. Chips were fractionated by the SO₂-ethanol-water (SEW) pulping process, also termed AVAP™ process by American Process Inc. (API). Pulping was done in a silicon oil bath using 6 bombs of 220 ml and 25 grams of oven dried of chips were placed in each bomb. The fresh fractionation liquor was prepared by injecting gaseous sulfur dioxide into a 55% (by volume) ethanol-water solution. Deionized water and ethanol ETAX A (96.1 v/v %) were used. The liquor-to-wood ratio used was 6 L/kg. Pulping was carried out in two different conditions. Fractionation conditions including the concentration of SO₂ in the liquor by weight, temperature and fractionation time including the heating-up period are shown in Table 1. Conditions were chosen so that the pulp obtained in the fractionation 2 has notably lower viscosity and cellulose degree of polymerization. In the further context, unfermented reference pulp samples are referred to as REF135 and REF150 according to the pulping temperatures, whereas the fermented pulps are referred to as FER135 and FER150.

TABLE 1
Fractionation conditions during the SEW pulping of
spruce chips into cellulosic fibers.

| Fractionation | | |
|---|---|---|
| conditions | Fractionation 1 | Fractionation 2 |
| Concentration of SO₂ | 12% | 3% |
| Temperature | 135° C. (±1° C.) | 150° C. (±1° C.) |
| Fractionation time | 80 min | 120 min |

After pulping, the bombs were rapidly removed from the oil bath and cooled in cold water. The pulp suspension from each bomb was poured in a washing sock and spent liquor was separated from pulp by squeezing. Pulp was then washed 2 times with 300 ml of 40% ethanol-water solution at 60° C. and finally 2 times with 3 L of deionized water at room temperature. A portion of the washed pulp was placed into the fermentation column whereas some pulp was kept as a reference sample with no further treatment.

Analyses of the Pulps Prior and after Fermentation

Homogenization of the untreated reference pulps and fermented pulps was carried out by disintegration at 1% consistency for 30,000 revolutions (apparatus according to ISO 5263). Disintegrated pulp was contained in a washing sock and excess water was filtered out. Filtrate was re-filtered through the fiber mat to diminish the loss of fines. Pulp was then air dried prior to further experiments.

The chemical composition of the pulps was determined after milling air dried pulps in a Wiley mill using a 30 mesh screen. Extractive content was determined according to SCAN-CM 49:03, using acetone as an extracting solvent. For the determination of lignin and structural carbohydrates, the procedure by NREL (NREL/TP-510-42618) was followed. Exception to the cited procedure was the determination of the acid soluble lignin (ASL) with an UV-Vis spectrophotometer at 205 nm wavelength, according to equation:

$$ASL = \frac{Absorbance \cdot {Volume}_{filtrate} \cdot Dilution}{{Absorbtivity}_{biomass} \cdot {ODW}_{pulp}} \cdot 100$$

where ODWₚᵤₗₚ-oven dry weight of the pulp sample. The absortivity value used was 128 L g⁻¹cm⁻¹, which is common for softwood species.

The intrinsic viscosity of pulp solutions in CED was analyzed according to SCAN-CM 15:99. Prior to the determination, the pulps REF150 and FER150 prepared with lower SO₂ charge (3%) and thereby having higher lignin content were exposed to chlorite delignification according to T230 om-66 (5 g pulp in 200 ml water+5 g NaClO₂+2 ml acetic acid at 70° C. for 5 min).

The cellulose degree of polymerization (DP) was calculated from the intrinsic viscosity according to the following equation (da Silva Perez and van Heiningen, 2002).

$$DP = {( \frac{1.65 [ η ] - {116 [ Hemi ]}_{pulp}}{{[ Cel ]}_{pulp}} )}^{1.111} ,$$

where η—intrinsic viscosity of pulps in CED, ml/g; [Hemi]ₚᵤₗₚ—hemicelluloses content of pulp (unit fraction) and [Cel]ₚᵤₗₚ—cellulose content of pulp (unit fraction). The cellulose content of the pulp was calculated using the equation (Janson, 1974).

[Cel]=[Glu]ₜₒₜ−[Man]/4.15,

where [Glu]ₜₒₜ—total glucan content of the pulp and [Man]—mannan content of the pulp. Glucan in hemicelluloses was calculated as the difference of the total glucan content and the cellulose content of the pulp.

### Substrate and Metabolite Analysis

Samples of 2 ml were centrifuged at 14 000 rpm for 5 min (Eppendorf, Centrifuge 5424) and the supernatant was recovered for analysis. Glucose, acetic acid, butyric acid, acetone, butanol, and ethanol concentrations were analysed by HPLC (Alliance, Waters 2690 and 2695 Separations Modules).

### Results

Two stage chemostat set up was constructed by connecting two bioreactor units with peristaltic pumps. The division between acidogenetic and solventogenetic phase in F1 and F2 bioreactor units was based on the pH difference (Table 1) according to Mutschlechner et al. (2000). The chemostat was running with two different dilution rates with glucose and sugar mixture substrates. The lower dilution rate was set to 0.05 and the higher 0.1 1/h, respectively. Above 0.1 1/h dilutions rates biomass started to slowly wash out indicating that the dilution rate maximum was surpassed. The analysed glucose substrate concentrations were 18.7 and 56.2 g/l. The sugar mixture substrate concentrations were glucose 6.3, arabinose 2.23, galactose 6.43, mannose 22.85 and xylose 7.51 g/l. This sugar composition was based on the average results obtained from spruce chips wood hydrolysis by water-ethanol-SO₂ cooking by Rakkolainen et al. (2010).

The results indicated that different pH value cannot completely separate the C. acetobutylicum metabolism into acidogenetic and solventogenetic phases. Acids and solvents were found from both F1 and F2 bioreactor units. Biomass increases with the increasing dilution rate on 18.7 g/l of glucose feed concentration. This indicated that cells cannot have their maintenance energy demand completely fulfilled at the dilution rate of 0.06 1/h. The metabolite production indicated that acetone and butanol were the main products from glucose and they can be found from both F1 and F2 units in these conditions.

TABLE 2
Two stage chemostat data of Clostridia acetobutylicum B5313
growing on glucose (18.7 or 56.2 g/L and sugar mixture medium;
glucose 6.3; arabinose 2.23; galactose 6.43; mannose 22.85;
xylose 7.51 g/l). The F1 and F2 were maintained at the different pH
value of 4.6 and 5.1 respectively in order to have cell metabolism
divided into acidogenetic and solventogenetic phase.

| | | Dry weight | Dry weight | | |
|---|---|---|---|---|---|
| | D | g/l | g/l | pH | pH |
| Substrate | 1/h | F1 | F2 | F1 | F2 |
| Glucose 18.7 g/l | 0.060 | 0.95 | 0.40 | 4.75 | 4.89 |
| | 0.097 | 1.74 | 1.34 | 4.74 | 5.05 |
| Glucose 56.2 g/l | 0.048 | 1.41 | 1.65 | 4.53 | 5.03 |
| | 0.095 | 1.45 | 1.82 | 4.55 | 5.05 |
| Sugar mix 45.3 g/l | 0.047 | 0.75 | 1.14 | 4.49 | 4.97 |

At the 18.7 g/L of glucose concentration both units F1 and F2 had approximately the same metabolite levels. This is due to the fact that cell suspension was continuously pumped from the F1 to F2 unit. However, only after the glucose concentration was increased to 56.2 g/L the butanol and acetone concentration increased correspondingly in the F2 unit indicating that solventogenetic phase required sufficient excess substrate concentration (Table 3). Mannose and glucose were consumed with the yields of 0.67 and 0.90 g/g respectively being the most preferred substrates. Consequently, arabinose and xylose were the least preferred substrates with the yields 0.04 and 0.11 g/g respectively (Table 4a-b). All mixed substrates were consumed simultaneously by the C. acetobutylicum, but no solvent production was detected in the chemostat conditions.

TABLE 3
Table 3. The metabolite production of Clostridia acetobutylicum B 5313 growing on glucose with 18.7 and 56.2 g/l
of concentrations and 45.3 g/l of sugar mixture medium on a two stage chemostat. The F1 and F2 units maintained
the cells in acidogenetic and solventogenetic phase respectively based on the different pH value. All the samples
were done in duplicates on two successive dates.

| Butyric | | Acetic | | | | | | | |
|---|---|---|---|---|---|---|---|---|---|
| Acid | | acid | | Acetone | | Butanol | | Ethanol | |
| g/l | g/l | g/l | g/l | g/l | g/l | g/l | g/l | g/l | g/l |
| F1 | F2 | F1* | F2* | F1 | F2 | F1 | F2 | F1 | F2 |
| 1.70 ± 0.04 | 1.63 ± 0.03 | 2.43 ± 0.06 | 2.40 ± 0.01 | 7.89 ± 0.12 | 7.81 ± 0.28 | 4.82 ± 0.06 | 4.78 ± 0.10 | 0.44 ± 0.05 | 0.38 ± 0.11 |
| 1.01 ± 0.13 | 1.34 ± 0.03 | 2.55 ± 0.01 | 2.86 ± 0.39 | 8.15 ± 0.08 | 8.25 ± 0.38 | 5.13 ± 0.14 | 4.76 ± 0.10 | 0.37 ± 0.02 | 0.51 ± 0.22 |
| 1.76 ± 0.10 | 2.62 ± 0.26 | 2.87 ± 0.12 | 3.46 ± 0.36 | 4.54 ± 0.22 | 9.32 ± 0.23 | 5.83 ± 0.59 | 8.57 ± 0.76 | nd | nd |
| 2.02 ± 0.01 | 2.83 ± 0.03 | 3.15 ± 0.06 | 3.86 ± 0.09 | 4.88 ± 0.00 | 8.51 ± 0.07 | 3.95 ± 0.09 | 6.27 ± 0.21 | nd | nd |
| nd | nd | 3.21 ± 0.04 | 3.27 ± 0.13 | nd | nd | nd | nd | nd | nd |

*MSS medium contained acetate 2.54 g/l,
**nd = not detected

Table 4a-b. Two stage chemostat data of Clostridia acetobutylicum B 5313 growing on a) glucose with 18.6 or 56.2 g/l and b) sugar mixture medium (glucose 6.3; arabinose 2.23; galactose 6.43; mannose 22.85; xylose 7.51 g/l).

The figures are presented as yields of g of consumed sugar/g of sugar concentration in the medium (Y s/s) and F1 and F2 refers to the fermentation units and F1+F2 is the total consumption of sugars.

TABLE 4a

| | | | Y s/s | Y s/s | Y s/s |
|---|---|---|---|---|---|
| | Concentration | D | Glu | Glu | Glu |
| Substrate | g/l | 1/h | F1 | F2 | F1 + F2 |
| Glu | 18.7 | 0.060 | 1.00 | 1.00 | 1.00 |
| Glu | 18.7 | 0.095 | 1.00 | 1.00 | 1.00 |
| Glu | 56.6 | 0.048 | 0.46 | 0.58 | 0.77 |
| Glu | 56.2 | 0.095 | 0.39 | 0.79 | 0.87 |

TABLE 4b

| Substrate | Concentration | D | Y s/s | Y s/s | Y s/s |
|---|---|---|---|---|---|
| g/l | g/l | 1/h | F1 | F2 | F1 + F2 |
| Glucose | 6.3 | 0.047 | 0.88 | 0.88 | 0.90 |
| Arabinose | 2.3 | 0.047 | 0.06 | 0.09 | 0.04 |
| Galactose | 6.4 | 0.047 | 0.07 | 0.13 | 0.19 |
| Mannose | 22.9 | 0.047 | 0.23 | 0.58 | 0.67 |
| Xylose | 7.5 | 0.047 | 0.05 | 0.06 | 0.11 |

TABLE 5
Cellulosic fibers prepared from spruce wood chips were loaded into a
column supported by a plastic net. Clostridia acetobutylicum B 5313
cells from bioreactor were loaded into the column by pumping cells
suspension through the column matrix. After cell saturation the feed was
changed into a) glucose with 18.7 or b) sugar mixture medium (glucose
6.3; arabinose 2.23; galactose 6.43; mannose 22.85; xylose 7.51 g/l). The
figures are presented as volumetric yields (Q_{BuOH} = g of butanol/l h).

| | | | | | | Yield |
|---|---|---|---|---|---|---|
| | Concentration | | D | | Q (BuOH) | (BuOH) |
| Substrate | g/l | Hours h | 1/h | pH | g/l h | g/g |
| GLU | 18.7 | 18 | 1.06 | 3.24 | 4.38 | 0.22 |
| GLU | 18.7 | 19 | 0.13 | 4.55 | 0.53 | 0.24 |
| MIX | 45.3 | 17 | 0.20 | 3.66 | 0.82 | 0.11 |
| MIX | 45.3 | 18 | 0.09 | 4.55 | 0.13 | 0.08 |

In order to achieve high volumetric productivity of ABE products the C. acetobutylicum cells from the F2 bioreactor were loaded into the column. Cellulosic fibers were used as a cell retaining matrix supported by a plastic net. The results indicate that the highest volumetric butanol production from glucose was 4.38 g/l h with the final concentration of 4.14 g/l (data not shown). The highest dilution rate (1.06 1/h) allowed rapid removal of butanol alleviating the inhibition effect on cell metabolism. The acetic and butyric acid concentrations were 1.19 g/l and 1.63 g/l respectively indicating that cells were actively producing acids (data not shown). The volumetric productivity and final concentration for the sugar mixture feed at the dilution rate of 0.2 1/h was 0.82 g/l h and 4.02 g/l (data not shown).

TABLE 6
Intrinsic viscosity of pulp in CED (ml/g) and cellulose degree of
polymerization (DP) before and after being used as a cell retaining matrix in the
ABE-column.REF135/150 is referring to the control samples before and
FER135/150 after the column trials.

| | REF135 | FER135 | REF150 | FER150 |
|---|---|---|---|---|
| Viscosity (ml/g) | 1125.4 ± 15.5 | 1036.4 ± 12.0 | 462.4 ± 4.2 | 455.0 ± 0.1 |
| DP | 5257.3 ± 81.1 | 4753.6 ± 61.5 | 1831.4 ± 18.6 | 1799.2 ± 0.7 |

The results on the viscosity and DP of cellulose show that C. acetobutylicum can degrade cellulosic fibers by producing extracellular cellulases in the column conditions. DP of cellulose was decreased by 9.6% in case of the pulp with higher viscosity, whereas the effect on pulp with lower viscosity was negligible (1.9%) (Table 6).

TABLE 7
The chemical composition of cellulosic fibers prior and after the ABE-
column runs.

| | REF135 | FER135 |
|---|---|---|
| | % | % |
| Extractives | 0.2 ± 0.0 | 0.2 ± 0.0 |
| Lignin | 5.6 ± 0.0 | 6.2 ± 0.1 |
| Acid insoluble | 5.2 ± 0.0 | 5.7 ± 0.1 |
| Acid soluble | 0.4 ± 0.0 | 0.5 ± 0.0 |
| Carbohydrates | 93.0 ± 0.1 | 92.1 ± 0.0 |
| Glucan | 83.2 ± 0.1 | 83.4 ± 0.1 |
| Mannan | 6.5 ± 0.0 | 5.8 ± 0.0 |
| Xylan | 3.3 ± 0.1 | 2.9 ± 0.0 |
| TOTAL | 98.9 ± 0.1 | 98.5 ± 0.1 |

The weight fraction of the total carbohydrates was decreased in the fermented pulp compared to the reference, whereas the proportional share of lignin was increased (Table 7). Mainly the sugars mannan and xylan, originated from hemicelluloses were consumed rather than glucan originating from cellulose.

Continuous Bio-Catalytic Conversion of Sugar Mixture to Acetone-Butanol-Ethanol by Immobilized C. acetobutylicum DSM 792

Glucose and D-xylose was purchased from VWR International, Finland, yeast extract, tryptone were purchased from Lab M Ltd, UK. Mannose, D-galactose, L-arabinose were purchased from Danisco, Finland. p-amino benzoic acid, MgSO₄, FeCl₃, NaMoO₄ and CaCl₂ were obtained from Fluka, Switzerland. L-cysteine hydrochloride and biotin were purchased from Sigma Aldrich, USA. K₂HPO₄, sodium sulphate, ZnSO₄, ZnSO₄, CuSO₄ and reinforced clostridia medium (RCM) were obtained from Merck, Germany. NaOH, HCl and H₂SO₄ were obtained from J.T. Baker, Holland. All the chemicals were analytical grade. C. acetobutylicum DSM 792 was obtained from DSMZ, Germany (German Collection of Microorganisms and Cell Cultures). Initially sporulated cells were activated by heat shock at 80° C. for 10 min. The activated spore culture (2.5 ml) was inoculated in 100 ml sterile RCM in 125 ml air tight, anaerobic glass bottles and grown for 20 h at 37° C. After 20 h, the inoculum was used for batch experiments (5% v/v) as well as for immobilization of matrix for continuous experiments. The inoculum medium (RCM) contained meat extract 10 g/l, peptone 5 g/l, yeast extract 3 g/l, D(+) glucose 5 g/l, starch 1 g/l, sodium chloride 5 g/l, sodium acetate 3 g/l and L-cysteine hydrochloride 0.5 g/l (final pH 6.8±0.2). The production medium contained (in g/l) glucose 60, magnesium sulphate 0.2, sodium chloride 0.01, manganese sulphate 0.01, iron sulphate 0.01, potassium dihydrogen phosphate 0.5, potassium hydrogen phosphate 0.5, ammonium acetate 2.2, biotin 0.01, thiamin 0.1 and p-aminobenzoic acid 0.1. Modified production medium contained sugar mixture (50 g/l) of glucose, mannose, arabinose, galactose and xylose instead of a single carbon source. It contained (in g/l) glucose 8.5, mannose 22.0, arabinose 2.3, galactose 4.5 and xylose 10.50. The medium was adjusted to pH 6.5 with HCl. After preparation, the medium was purged with oxygen free nitrogen and autoclaved at 10⁵ Pa (121° C.) for 20 min and cooled.

In the present study, we chose support materials like wood pulp, loofa sponge, coconut fibers, wood chips and sugarcane baggase. The wood pulp fibers and wood chips were obtained from Department of Forest Products Technology, Aalto University School of Science and Technology, Espoo, Finland. The matrices were cut into 3-5 mm from their raw sources. They were washed with water for three times and dried in oven at 70° C. All the immobilization materials were evaluated for maximum solvent production in batch mode. Processed matrices were added to production medium at ratio of 1:4 v/v in 125 ml air tight bottles. It was purged with nitrogen and autoclaved at 10⁵ Pa (121° C.) for 20 min and cooled. It was inoculated (5% v/v) with 20 h actively growing seed culture and incubated for 110 h at 37° C. The wood pulp soaked in excess of water was distributed evenly on plastic mesh and rolled to remove excess of water. The plastic mesh was used for holding the wood pulp. The roll was put into a jacketed glass column and used for immobilization of cells. The column was filled with 70% ethanol and kept for 24 h for sterilization. The ethanol was replaced with deoxygenated production medium after 24 h. The inoculum was pumped into the column and re-circulated for 24 h for cell adsorption and growth.

After immobilizing cells, the production medium was continuously fed to the immobilized cell reactor at different dilution rates. The dilution rate was altered whenever a steady state was reached in terms of production of solvents and acids and use of substrate. After changing the dilution rate, sufficient time was allowed to pass in order to reach a new steady state before samples were taken from the top of the column and centrifuged at 15000 rpm for 5 min and supernatants were used for the substrate and product analysis. The column temperature was maintained at 37° C. by continuously circulating water through the jacket.

The solvents and acids were quantified by using gas chromatography. The gas chromatograph (Hewlett Packard series 6890) equipped with a flame ionization detector was used. Separation took place in DB-WAXetr capillary column (30 m×0.32 mm×1 μm) from Agilent Technologies, Finland. The injector temperature was 200° C. and detector temperature was 250° C. The injector volume was 10 μl. Glucose, mannose, arabinose, galactose and xylose were determined by high-performance liquid chromatography (Biorad Laboratories, Richmond, Calif.), equipped with an Inores S 259-H column (Inovex, Vienna, Austria) packed with Inores cation exchanger (particle size, 9 mm). The column was heated at 70° C., and the eluent (0.01M H₂SO₄) was circulated with a flow rate of 0.60 mL min⁻¹. A cellobiose (Roth, Karlsruhe, Germany) solution was added to the samples as an internal standard. A refractive index detector (model 1755; Bio-Rad) was used for quantification.

Calculation of Bioprocess Parameters.

Dilution rate in h⁻¹ was calculated as flow rate divided by the working volume of the column. The overall solvent productivity in g/(I·h) during continuous cultivation of solvent-producing clostridia was expressed as g/l of total solvents multiplied by dilution rate (h⁻¹). Solvent yield was calculated by dividing total solvents in g/l by utilized substrate in g/l.

Cell immobilization is often used to improve the performance of traditional continuous fermentation process by increasing the amount of cells per bioreactor volume, and cell deposition on support matrix. Cell immobilization through adsorption provides a direct contact between nutrients and the immobilized cells. This technique involves the transport of the cells from the bulk phase to the surface of support, followed by the adhesion of cells, and subsequent colonization of the support surface. Both electrostatic and hydrophobic interactions govern the cell-support adhesion, which is the key step in controlling the cell immobilization on the support

It was found that addition of support matrix helps in improving substrate consumption and conversion to solvents. Coconut fibers and wood pulp were found to be the most promising support matrices. The maximum solvent concentration of 18.88 g/l and 18.60 g/l was obtained with wood pulp and coconut fiber respectively, after 110 h of fermentation as compared to 8.18 g/l with control. Among all the support matrices, wood pulp and coconut fibers containing bottle complete glucose consumption was observed. In control experiment i.e. without adding any support matrix maximum glucose consumption was approximately 65%.

In summary: Continuous production of acetone, n-butanol and ethanol (ABE) was successfully carried out using immobilized Clostridium acetobutylicum DSM 792. Wood pulp fibers were used to immobilize C. acetobutylicum cells. Initially, different lignocellulosic materials were evaluated as an immobilization matrix for maximum production of ABE. Coconut fibers and wood pulp fibers were found to be promising. Further, wood pulp was used as cell holding material in column reactor for continuous production of ABE mixture. Glucose as well as sugar mixture (glucose, mannose, galactose, arabinose and xylose) identical to lignocellulosic hydrolysate was used as substrate for the production of solvents. The effect of dilution rate on solvent production was studied during continuous (nearly 25 days) operation. The maximum total solvent concentration of 14.32 g/l was obtained at a dilution rate of 0.22 h⁻¹ with glucose as substrate as compared to 12.64 g/l at 0.5 h⁻¹ dilution rate with sugar mixture. The maximum solvent productivity (13.66 g/l·h) was obtained at dilution rate of 1.9 h⁻¹ with glucose as substrate whereas solvent productivity (12.14 g/l·h) was obtained at dilution rate of 1.5 h⁻¹ with sugar mixture. The immobilized column reactor was found to be suitable for continuous production of ABE using sugar mixture.

Continuous Acetone-Butanol-Ethanol Fermentation Using SO₂-Ethanol-Water Spent Liquor from Spruce

The cost of biomass is an important parameter for the economical production of butanol. Lignocellulose biomass is considered as the cheapest as well as sustainable feedstock. Continuous production of acetone, butanol and ethanol (ABE) was studied using SO₂-ethanol-water (SEW) spent liquor. Initially, batch experiments were performed using spent liquor to check the suitability for production of ABE. The overall study will serve the concept of forest biorefinery by using forest biomass for production of valuable compounds. The spent liquor from SEW fractionation process was successfully used for ABE solvent production. A continuous ABE solvent production process using an efficient column reactor with wood pulp fibers as an immobilization material and SEW spent liquor as substrate is developed. The bioreactor was operated for nearly 20 days in continuous flow mode. The use of cheap substrate along with continuous mode production makes the process industrially attractive. Further, we developed method for efficient utilization of spent broth to make process more economical.

Glucose was purchased from VWR International, Finland. p-amino benzoic acid, MgSO₄, FeSO₄, NaCl were obtained from Fluka, Switzerland. L-cysteine hydrochloride and biotin were purchased from Sigma Aldrich, USA. K₂HPO₄, KH₂PO₄, MnSO₄, ammonium acetate, and reinforced clostridia medium (RCM) were obtained from Merck, Germany. NaOH and HCl were obtained from J.T. Baker, Holland. All the chemicals were analytical grade. Amberlite XAD-4 resin was a kind gift from Rohm and Haas, France.

C. acetobutylicum DSM 792 was obtained from DSMZ, Germany (German Collection of Microorganisms and Cell Cultures). Initially sporulated cells were activated by heat shock at 80° C. for 10 min. The activated spore culture (2.5 ml) was inoculated in 100 ml sterile RCM in 125 ml air tight, anaerobic glass bottles and grown for 20 h at 37° C. After 20 h, the inoculum was used for batch experiments (5% v/v) as well as for immobilization of matrix for continuous experiments.

The production of solvents was studied using SEW spent liquor. The liquor was supplemented with the medium components reported by Tripathi et al. (2010). The supplement contained (in g/l) magnesium sulphate 0.2, sodium chloride 0.01, manganese sulphate 0.01, iron sulphate 0.01, potassium dihydrogen phosphate 0.5, potassium hydrogen phosphate 0.5, ammonium acetate 2.2, biotin 0.01, thiamin 0.1 and p-aminobenzoic acid 0.1. The glucose was added as and when mentioned. The pH was adjusted to 6.5 with HCl. After preparation, the medium was purged with oxygen free nitrogen and autoclaved at 10⁵ Pa (121° C.) for 20 min and cooled. RCM was used for inoculum preparation. The RCM contained meat extract 10 g/l, peptone 5 g/l, yeast extract 3 g/l, D(+) glucose 30 g/l, starch 1 g/l, sodium chloride 5 g/l, sodium acetate 3 g/l and L-cysteine hydrochloride 0.5 g/l (final pH 6.8±0.2).

The SEW spent liquor was produced and conditioned as reported by Sklavounos et al. (2011). The fractionation of spruce wood chips was carried out using SEW liquor with liquor-to-wood ratio of 6:1 kg. The spent SEW liquor was processed with a sequence of conditioning steps including evaporation, steam stripping, liming and catalytic oxidation for making it fermentable. In SEW liquor, the acetic acid concentration was 1.5 g/l (1.0 g/100 g O.D. wood) and the formic acid concentration was close to zero. The furfural and hydroxy methyl furfural (HMF) concentrations in SEW liquor were 0.7 and 0.2 g/l, respectively. The SO₂ level was sufficiently minimized to 6 mg/l. The total sugar concentration in final liquor was approximately 111.0 g/l. The individual sugar concentration was (in g/l) glucose 18.8, mannose 52.3, galactose 9.7, arabinose 5.4 and xylose 24.8. The obtained liquor was further treated with anion exchange resin (Amberlite XAD-4). The resin was removed by filtration. The pH of spent liquor was finally adjusted to 6.5 with Ca(OH)₂ before adding the medium components.

The effect of dilution of SEW liquor was studied on production of solvents. The SEW spent liquor was diluted as 2 fold, 4 fold and 8 fold with water to make it suitable for growth and fermentation of clostridia. The diluted liquor (8 fold) was also tried as an inoculum medium. All the production medium components except carbon source were supplemented to the spent liquor. The effect of supplementing the extra glucose (15, 25 and 35 g/l) to the 4 fold diluted SEW liquor was also studied. The batch experiments were carried out in 125 ml screw cap bottles with 50 ml production medium. It was purged with nitrogen and autoclaved at 10⁵ Pa (121° C.) for 20 min and cooled. It was inoculated (5% v/v) with 20 h actively growing seed culture and incubated for 96 h at 37° C. The optimized medium composition was further tested for continuous operation. All the experiments were done at least in triplicates and values are given as means and standard deviation was established using Microsoft Excel software.

The wood pulp was used as an immobilization material. The column was filled with 70% ethanol and kept for 24 h for sterilization. The inoculum was pumped into the column and re-circulated for 24 h for cell adsorption and growth.

After immobilizing cells, the SEW spent liquor was continuously fed to the immobilized cell reactor at different dilution rates. The dilution rate was altered whenever a steady state was reached in terms of production of solvents and acids. After changing the dilution rate, 2 volume changes was allowed to pass in order to reach a new steady state before samples were taken from the top of the column and centrifuged at 15000 rpm for 5 min. Supernatants were used for the substrate and product analysis. The column temperature was maintained at 37° C. by continuously circulating water through the jacket.

The SB collected after continuous fermentation contained residual sugars and some medium components. The feasibility of SB as production medium was checked in batch experiments after removing the solvents produced. The solvents produced were removed by nitrogen gas purging. The SB was used as such or supplemented with production medium components. The batch experiments were carried out in 125 ml screw cap bottles with 50 ml production medium as reported in earlier section. The solvents and acids were quantified by using gas chromatography. Glucose, mannose, arabinose, galactose and xylose were determined by high-performance liquid chromatography.

In Summary: Maximum concentration of total ABE was found to be 8.79 g/l using 4 fold diluted SEW liquor supplemented with 35 g/l of glucose. The effect of dilution rate on solvent production, productivity and yield was studied in column reactor consisting of immobilized Clostridium acetobutylicum DSM 792 on wood pulp. Total solvent concentration of 12 g/l was obtained at a dilution rate of 0.21 h⁻¹. The maximum solvent productivity (4.86 g/l·h) with yield of 0.27 g/g was obtained at dilution rate of 0.64 h⁻¹. Further, to increase the solvent yield, the unutilized sugars were subjected to batch fermentation after taking out solvents produced. We successfully used SEW liquor for batch and continuous production of ABE solvents.

### REFERENCES

Bahl, H., Andersch, W. and Gottschalk, G. (1982) Continuous production of acetone and butanol by Clostridium acetobutylicum in a two-stage phosphate limited chemostat. Eur. J. Appi. Microbiol. Biotechnol. 15:201-205.
Berezina, O. V., Sineoky, S. P., Velikodvorskaya, G. A., Schwarz, W. H. and Zverlov, V. V. (2008) Extracellular glycosyl hydrolase activity of the Clostridium strains producing acetone, butanol, and ethanol. Appl. Biochem. Microbiol. 44:42-47.
Berezina, O. V., Zakharova, N. V., Brandt, A., Yarotsky, S. V., Schwarz, W. H. and Zverlov, V. V. (2010) Reconstructing the clostridial n-butanol metabolic pathway in Lactobacillus brevis. Appl Microbiol Biotechnol. 87:635-646.
Cornillot, E., Nair, R. V., Papoutsakis, E. T. and Soucaille, P. (1997) The genes for butanol and acetone formation in Clostridium acetobutylicum ATCC 824 reside on a large plasmid whose loss leads to degeneration of the strain. Journal of Bacteriology. 179(17), 5442-5447.
Ennis, B. M. and Maddox, I. S. (1989) Production of solvents (ABE fermentation) from whey permeate by continuous fermentation in a membrane bioreactor. Bioprocess Eng. 4: 27-34.
Ezeji T. and Blaschek H. P. (2008) Fermentation of dried distillers' grains and solubles (DDGS) hydrolysates to solvents and value-added products by solventogenic Clostridia. Bioresource Technology. 99(12): 5232.
Harris, L. M., Welker, N. E., Papoutsakis, E. T. (2002) Northern, morphological, and fermentation analysis of spo0A inactivation and overexpression in Clostridium acetobutylicum ATCC 824. Journal of Bacteriology. 184(13), 3586-3597.
Janson, J. (1974) Analysis of the polysaccharides in wood and pulp. Faserforschung and Textiltechnik. 25(9), 375-382.
Lin, Y. L. and Blaschek, H. P. (1983) Butanol production by a butanol-tolerant strain of Clostridium acetobutylicum in extruded corn broth. Applied and Environmental Microbiology. 45(3): 966-73.
Liubimova I. K., Velikaia, M. A., Lukina, G. P., Abilev, S. K. and Livshits, V. A. (1993) Biosynthesis of solvents by the mutants of Clostridium acetobutylicum, resistant to 2-deoxy-D-glucose. Russian Journal of Biotechnology. 8: 10-12.
López-Contreras, A. M., Gabor, K, Martens, A. A., Renckens, B. A. M., Claassen, P. A. M., van der Oost, J. and de Vos, W. M. (2004) Substrate-induced production and secretion of cellulases by Clostridium acetobutylicum. Applied and Environmental Microbiology. 70(9): 5238-5243.
Mitchell, W. J., (1998) Physiology of carbohydrate to solvent conversion by Clostridia. Adv. Microb. Physiol. 39, 31-130.
Mutschlechner O., Swoboda H. and Gapes J. R. (2000) Continuous two-stage ABE-fermentation using Clostridium beijerinckii NRRL 8592 operating with a growth rate in the first stage vessel close to its maximal value. J. Mol. Microbiol. Biotechnol. 2(1):101-105.
Nimcevic D., Schuster M. and Gapes J. R. (1998) Solvent production by Clostridium beijerinckii NRRL 8592 growing on different potato media. Appl. Microbiol. Biotechnol. 50: 426-428.
Qureshi N., Lai, L. L. and Blaschek, H. P. (2004) Scale-up of a high productivity continuous biofilm reactor to produce butanol by adsorbed cells of Clostridium beijerinckii. Food and Bioproducts Processing. 82(C2): 164-173.
Rakkolainen, M., Iakovlev, M., Terasvuori, A-L., Sklavounos, G. Jurgens, Granström, T. B. and van Heiningen, A. (2010) SO₂-ethanol-water fractionation of forest biomass and implications for biofuel production by ABE fermentation. Cellulose Chemistry and technology (in press).
da Silva Perez, D. and van Heiningen, A. R. P. (2002) Determination of cellulose degree of polymerization in chemical pulps by viscosimetry. Seventh European Workshop on Lignocellulosics and Pulp (EWLP, Proceedings), pp. 393-396.
Tashiro, Yukihiro; Takeda, Katsuhisa; Kobayashi, Genta and Sonomoto, Kenji (2005) High production of acetone-butanol-ethanol with high cell density culture by cell-recycling and bleeding. Journal of Biotechnology. 120(2), 197-206.
Thormann, K., Feustel, L., Lorenz, K., Nakotte, S, and Durre, P. (2002) Control of butanol formation in Clostridium acetobutylicum by transcriptional activation. Journal of Bacteriology. 184(7), 1966-1973.
Walton, M. T. and Martin, J. L. (1979) Production of butanol-acetone by fermentation. In H. J. Peppler and D. Perlman (Eds.) Microbial Technology 2^{nd} ed. Academic Press. New York, N.Y. 1:187-209.
Zverlov, V. V., Berezina, O., Velikodvorskaya, G. A. and Schwarz, W. H. (2006) Bacterial acetone and butanol production by industrial fermentation in the Soviet Union: use of hydrolyzed agricultural waste for biorefinery. Appl. Microbiol. Biotechnol. 71, 587-597.

1. A cell retaining biomatrix (CRB) in the form of sheet, mat or strip for producing organic solvents and alcohols by microbe(s) from different substrates, wherein the cell retaining biomatrix (CRB) comprises:
cellulosic fibers, selected from the group consisting of wood cellulosic fibers, pulp cellulosic fibers, vegetable cellulosic fibers and cellulosic fibers originated from vegetable peels,
microbes, which have been immobilized into said cellulosic fibers, and which can save their biological activity in the cell retaining biomatrix (CRB); and
a support structure or an effluent or both a support structure and an effluent.

2. The cell retaining biomatrix (CRB) according to claim 1, wherein said biologically active microbes can save their biological activity at least for 14 days in the cell retaining biomatrix (CRB).

3. (canceled)

4. (canceled)

5. The cell retaining biomatrix (CRB) according to claim 1, wherein said microbe is selected from the group consisting of Clostridia species, and Lactobacillus species.

6. A bio-column (BC) comprising a cell retaining biomatrix (CRB) according to claim 1.

7. An apparatus for producing organic solvents and alcohols from different substrates by microbe(s), comprising at least the bio-column (BC) according to claim 6.

8. The apparatus according to claim 7, further comprising a separate or an integrated cell growing unit (F1) having a feeding device for feeding a first substrate (SU1) and an adjusting device for controlling growth conditions of cells of selected microbe(s) in a first solution (SO1) in said integrated cell growing unit (F1).

9. The apparatus according to claim 8, further comprising a separate or an integrated fermentation adjusting unit (F2) having a feeding device for feeding cells and/or the first solution (SO1) from said integrated cell growing unit (F1) and an adjusting device for adjusting condition in a second solution (SO2) to favor production of organic solvents and alcohols by cells of selected microbe(s).

10. The apparatus according to claim 8, further comprising a separate or an integrated solution recovering unit for recovering a third solution (SO3) from the bio-column (BC), said third solution comprising organic solvents and alcohols.

11. The apparatus according to claim 8, further comprising a cell return unit for recovering cells, solution and/or fibers originated from the bio-column (BC), from the substrate (SU1, SU2) and/or from the solution recovering unit, and optionally for returning at least part of those cells to the integrated cell growing unit, to the fermentation adjusting unit, and/or to the bio-column (BC).

12. A method for producing organic solvents and alcohols from different substrates comprising at least the following steps:
feeding a substrate (SU1, SU2) to the cell retaining biomatrix (CRB) according to claim 1,
producing organic solvents and alcohols with microbes by feeding the substrate to the cell retaining biomatrix (CRB), and
recovering a solution comprising organic solvents and alcohols.

13. The method according to claim 12, further comprising the step of:
growing cells of microbe(s) in a first solution (SO1) by feeding the first substrate (SU1) and adjusting a condition of said first solution (SO1) for optimal growth conditions of cells of microbes.

14. The method according to claim 12, wherein amount of cells is controlled by measuring optical density value of the solutions (SO1, SO2, SO3).

15. The method according to claim 12, wherein the substrate (SU1, SU2) is selected from the group consisting of monomeric and oligomeric sugars, substrate originated from wood biomass and lignocellulosic biomass, and substrate originated from vegetable peels, such as sulphite spent liquor (SSL), POME, and EFB.

16. The method according to claim 12, wherein the growth conditions are optimized by controlling pH value, growth rate, feeding rate, temperature, sugar composition, or substrate concentration.

17. An organic solvent or an alcohol, produced by a cell retaining biomatrix (CRB) according to claim 1, wherein at least one substrate is selected from the group consisting of monomeric and oligomeric sugars, substrate originated from wood biomass and lignocellulosic biomass; and substrate originated from vegetable peels, such as sulphite spent liquor (SSL), POME, and EFB.

18. (canceled)

19. The cell retaining biomatrix (CRB) of claim 1, wherein vegetable cellulosic fibers are selected from the group consisting of mechanical pulp, dissolving pulp, and lignocellulosic fibers.

20. The cell retaining biomatrix (CRB) according to claim 5, wherein the Clostridia species include C. acetobutylicum, C. butyricum, C. beijerinckii, C. saccharobutylacetonicum, C. saccharobutylicum, and C. saccharoperbutylacetonicum and the Lactobacillus species include L. plantarum, L. brevis, L. fermentum, L. sanfranciscensis, L. buchneri, L. collinoides, L. rhamnosus and L. bulgaricus.

21. The method according to claim 15, wherein the substrate originated from vegetable peels is selected from the group consisting of sulphite spent liquor (SSL), POME, and EFB.

22. The organic solvent or alcohol of claim 17, wherein the solvent or alcohol is acetone, butanol, ethanol or isopropanol.

23. The method of claim 13, wherein the method comprises a step of adjusting a condition of grown cells in the second solution (SO2) to favor production of organic solvents and alcohols by cells of microbes.
